(19) United States
(12) Patent Application Publication
(10) Pub. No.: US 2023/0223128 A1
(43) Pub. Date: Jul. 13, 2023

(54) INFORMATION PROCESSING APPARATUS AND INFORMATION PROCESSING METHOD

(71) Applicant: OLYMPUS CORPORATION, Tokyo (JP)

(72) Inventors: Yusuke KATO, Tokyo (JP); Hidenori YASUOKA, Tokyo (JP); Syuta KOIZUMI, Tokyo (JP); Megumi NAGASAWA, Tokyo (JP); Kazuyoshi TAMURA, Tokyo (JP)

(73) Assignee: OLYMPUS CORPORATION, Tokyo (JP)

(21) Appl. No.: 18/118,962

(22) Filed: Mar. 8, 2023

Related U.S. Application Data
(63) Continuation of application No. PCT/JP2020/034371, filed on Sep. 10, 2020.

(51) Int. Cl. G16H 20/40; G16H 50/30

(52) U.S. Cl. CPC G16H 20/40; G16H 50/30

(57) ABSTRACT

A first examination identification unit identifies examination data including predetermined diagnosis data from among multiple pieces of examination data held in a recorder. A second examination identification unit identifies respective pieces of examination data of multiple examinations performed on a same patient after an examination date included in the examination data identified by the first examination identification unit from among the multiple pieces of examination data held in the recorder. An information acquisition unit acquires, based on multiple pieces of examination data identified by the first examination identification unit and the second examination identification unit, a period from the examination date included in the examination data identified by the first examination identification unit until the complete cure of the patient and the number of examinations performed until the complete cure of the patient. A derivation unit derives an appropriate examination interval and an appropriate number of examinations based on the period and the number of examinations acquired by the information acquisition unit from the plurality of pieces of examination data of the patient.

This application is based upon and claims the benefit of priority from the International Application No. PCT/JP2020/034371, filed on Sep. 10, 2020, the entire contents of which are incorporated herein by reference.

### BACKGROUND

### 1. Field of the Disclosure

The present disclosure relates to a medical support technology for managing examination data.

### 2. Description of the Related Art

At medical facilities, patients with a possibility of cancer recurrence are treated as high-risk patients, and endoscopic examinations are performed on such patients on a regular basis for follow-up. For example, patients who have been diagnosed with early-stage cancer through endoscopic examinations and have undergone endoscopic mucosal resection (EMR) or endoscopic submucosal dissection (ESD) are treated as high-risk patients with a high possibility of cancer recurrence and are subject to follow-up. Conventionally, examination schedules for high-risk patients are determined by the attending doctors, and the intervals between examinations and the number of examinations vary from doctor to doctor.

Patent Document 1 discloses a medical service support apparatus that calculates, for every examinee suffering from a specific disease, the number of days for the difference between the examination date of examination data in which the examinee has been diagnosed as normal and the subsequent examination date of examination data in which the examinee has been diagnosed with disease and performs a statistical process on the number of days so as to thereby determine a recommended examination interval for the disease.

[Patent Document 1] PCT International Publication No. WO2011/048812

High-frequency examinations on high-risk patients allows for early detection of cancer recurrence. However, high-frequency examinations impose a burden on the patients. Further, for medical facilities, conducting high-frequency examinations on one patient may result in wasting medical resources. Therefore, endoscopic examinations are desirably performed at an appropriate frequency and for an appropriate number of times for patients under follow-up.

In this background, a purpose of the present disclosure is to provide a technology for supporting endoscopic examinations.

An information processing apparatus according to one embodiment of the present disclosure is an information processing apparatus that is connected to a recorder that holds a plurality of pieces of examination data including an examination date and diagnostic data of an endoscopic examination that has been performed, including: a first examination identification unit that identifies examination data including predetermined diagnosis data from among a plurality of pieces of examination data held in the recorder; a second examination identification unit that identifies respective pieces of examination data of a plurality of examinations performed on a same patient after an examination date included in the examination data identified by the first examination identification unit from among the plurality of pieces of examination data held in the recorder; an information acquisition unit that acquires, based on a plurality of pieces of examination data identified by the first examination identification unit and the second examination identification unit, a period from the examination date included in the examination data identified by the first examination identification unit until the complete cure of the patient and the number of examinations performed until the complete cure of the patient; and a derivation unit that derives an appropriate examination interval and an appropriate number of examinations based on the period and the number of examinations acquired by the information acquisition unit from the plurality of pieces of examination data of the patient.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings that are meant to be exemplary, not limiting, and wherein like elements are numbered alike in several figures, in which:

FIG. 1 is a diagram showing an example of the configuration of a medical system according to an embodiment;

FIG. 2 is a diagram showing an example of examination data stored in a recorder;

FIG. 3 is a diagram showing an example of a time series of examination data of a patient;

FIG. 4 is a diagram showing an example of a time series of examination data of a patient;

FIG. 5 is a diagram showing an example of a time series of examination data of a patient;

FIG. 6 is a flowchart for generating a patient's examination schedule; and

FIG. 7 is a diagram showing an example of a notification screen displayed on a report entry screen.

### DETAILED DESCRIPTION

The disclosure will now be described by reference to the preferred embodiments. This does not intend to limit the scope of the present disclosure, but to exemplify the disclosure.

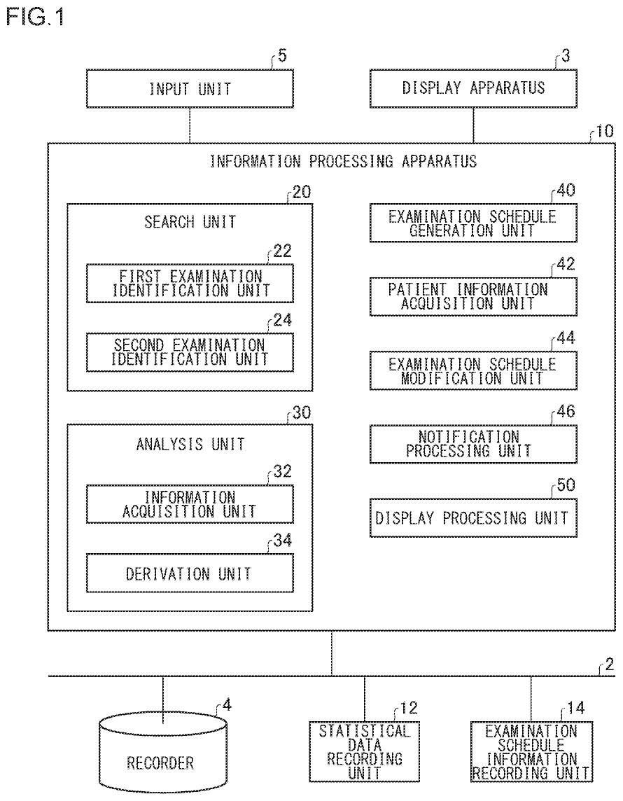

FIG. 1 shows a configuration example of a medical system 1 according to an embodiment of the present disclosure. The medical system 1 according to the embodiment is installed in a medical facility such as a hospital and includes an information processing apparatus 10, a recorder 4, a statistical data recording unit 12, and an examination schedule information recording unit 14. The information processing apparatus 10, the recorder 4, the statistical data recording unit 12, and the examination schedule information recording unit 14 are communicatively connected by a network 2 such as a local area network (LAN).

The recorder 4 holds a plurality of pieces of examination data including information on endoscopic examinations performed on patients. The recorder 4 may be an auxiliary storage device such as a hard disk drive (HDD) or solid state drive (SSD). The statistical data recording unit 12 and the examination schedule information recording unit 14 may be provided independently of the recorder 4, or may be configured as a recording area in the recorder 4. In the configuration example shown in FIG. 1, the recorder 4 is connected to the network 2 in the medical facility. Alternatively, the recorder 4 may be configured as a cloud server and connected to the information processing apparatus 10 via the Internet.

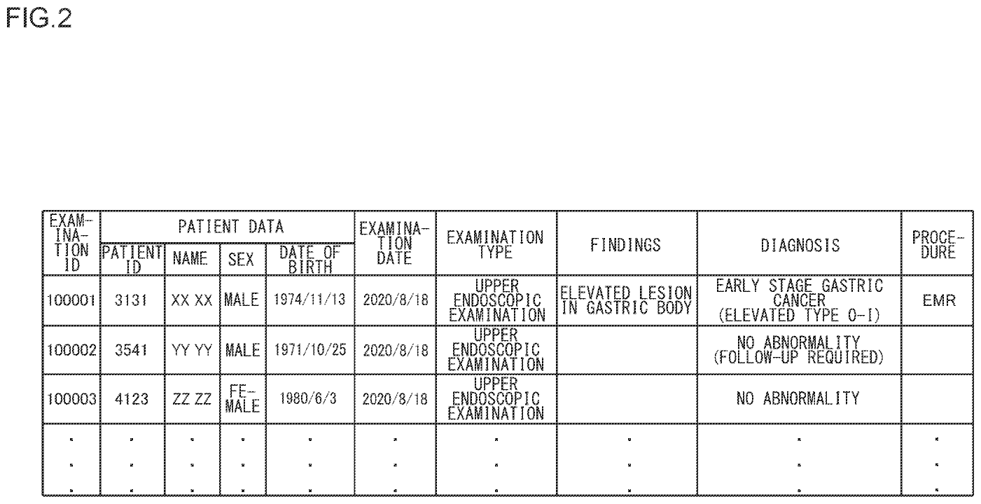

FIG. 2 is a diagram showing an example of examination data stored in the recorder 4. In the medical system 1, one piece of examination data is generated for one endoscopic examination. The format of the examination data includes the following items: examination ID, patient data, examination date, examination type, finding data, diagnosis data, and procedure data. The patient data includes patient ID, name, sex, and date of birth.

Examination data for an examination assigned with an examination ID “100001” includes information on an endoscopic examination performed on a patient “3131”. In this examination, an elevated lesion was observed in the gastric body and diagnosed with early stage gastric cancer, and endoscopic mucosal resection (EMR) was performed. In the medical system 1, patients who have undergone procedures such as EMR are treated as high-risk patients with a high possibility of cancer recurrence and are subject to follow-up.

In the medical system 1, in order to efficiently perform follow-up of a high-risk patient, the information processing apparatus 10 has a function of deriving an appropriate examination interval and an appropriate number of examinations from the past examination data accumulated in the recorder 4, based on the date when an endoscopic examination by which the patient was recognized as a high-risk patient has been performed (hereinafter also referred to as “reference date”). The examination interval means a period between two examinations.

Examination data for an examination assigned with an examination ID “100002” includes information on an endoscopic examination performed on a patient “3541”. No abnormal finding was found in this examination, but the patient was placed under “follow-up required”. In this example, the patient “3541” has been identified as a high-risk patient in the past, and until examinations are completed for the derived appropriate number of times, the “follow-up required” status is maintained even if there are no abnormal findings. Therefore, the patient “3541” will be scheduled for the next examination after the completion of the examination on 8/18/2020 based on the derived appropriate examination interval.

Examination data for an examination assigned with an examination ID “100003” includes information on an endoscopic examination performed on a patient “4123”. In this examination, there were no abnormal findings, and the patient was not placed under “follow-up required”. During the follow-up period, a high-risk patient undergoes examinations for the appropriate number of times at appropriate examination intervals, and follow-up is terminated if no abnormal findings are found in the last examination. This patient “4123” is no longer under the follow-up and is not scheduled for the next examination.

Referring back to FIG. 1, the information processing apparatus 10 according to the embodiment includes a communication circuit (not shown) that communicably connects the recorder 4, the statistical data recording unit 12, and the examination schedule information recording unit 14. Furthermore, the information processing apparatus 10 includes a search unit 20, an analysis unit 30, an examination schedule generation unit 40, a patient information acquisition unit 42, an examination schedule modification unit 44, a notification processing unit 46, and a display processing unit 50. The search unit 20 has a first examination identification unit 22 and a second examination identification unit 24, and the analysis unit 30 has an information acquisition unit 32 and a derivation unit 34.

The configuration shown in FIG. 1 is implemented by hardware such as an arbitrary processor, memory, auxiliary storage, or other LSIs and by software such as a program or the like loaded into the memory. The figure depicts functional blocks implemented by the cooperation of hardware and software. Thus, a person skilled in the art should appreciate that there are many ways of accomplishing these functional blocks in various forms in accordance with the components of hardware only, software only, or the combination of both.

The information processing apparatus 10 has a function of deriving an appropriate examination interval and an appropriate number of examinations for the purpose of performing follow-up of high-risk patients by statistically processing multiple pieces of past examination data recorded in the recorder 4. The information processing apparatus 10 may calculate an examination interval and the number of examinations according to an entered diagnosis when a doctor enters diagnosis data, etc., for a patient, or may calculate the examination interval and the number of examinations according to various types of diagnoses as statistical data at a predetermined time and register the calculated examination interval and the calculated number of examinations in the statistical data recording unit 12 in advance. In the latter case, when a doctor enters diagnosis data, etc., for a patient, the information processing apparatus 10 acquires an examination interval and the number of examinations according to an entered diagnosis by reading the examination interval and the number of examinations from the statistical data recording unit 12. In the embodiment, an appropriate examination interval and an appropriate number of examinations according to a diagnosis are derived by the search unit 20 and the analysis unit 30.

The search unit 20 has a function of accessing the recorder 4 and searching for examination data held in the recorder 4. The first examination identification unit 22 identifies examination data including predetermined diagnosis data showing that there is a possibility of cancer reoccurrence from among multiple pieces of examination data held in the recorder 4. In the medical system 1, a high-risk patient with a possibility of cancer recurrence is defined as a patient who has been diagnosed with early-stage cancer and who has undergone a procedure to remove the lesion. In the embodiment, as examination data indicating that there is a possibility of cancer recurrence, the first examination identification unit 22 searches for examination data that includes diagnosis data of “early gastric cancer” and procedure data for removing the lesion. In another example, the first examination identification unit 22 may search for examination data that meets other conditions for identifying the patient as a high-risk patient.

The second examination identification unit 24 identifies respective pieces of examination data of multiple examinations performed on the same patient after an examination date (reference date) included in the examination data identified by the first examination identification unit 22 from among the multiple pieces of examination data held in the recorder 4. In other words, the second examination identification unit 24 identifies examination data of examinations performed after the reference date for the purpose of performing follow-up. The following shows an example of a time series of examination data for each patient.

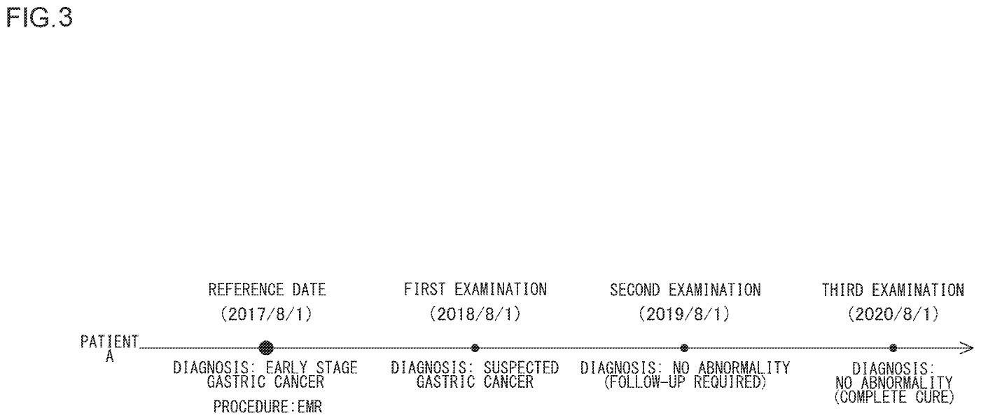

FIG. 3 shows a time series of examination data of Patient A. The first examination identification unit 22 identifies examination data that includes “early gastric cancer” diagnosis data and in which procedure data performed on the lesion is recorded. In the example shown in FIG. 3, the first examination identification unit 22 identifies examination data of Patient A whose examination date is 2017/8/1, and “EMR” is recorded in this examination data as the procedure performed on the lesion.

The second examination identification unit 24 uses the examination date (2017/8/1) of the examination data identified by the first examination identification unit 22 as a reference date and identifies respective pieces of examination data of multiple examinations performed on Patient A after the reference date. In the example shown in FIG. 3, the second examination identification unit 24 identifies examination data of the following three examinations.

(1) First Examination

Examination date: 2018/8/1

Diagnosis: Suspected gastric cancer

(2) Second Examination

Examination date: 2019/8/1

Diagnosis: No abnormality (follow-up required)

In the examination data of the second examination, “follow-up required” diagnosis data indicates that the follow-up is still ongoing and that the doctor has not yet diagnosed a complete cure for Patient A.

(3) Third Examination

Examination date: 2020/8/1

Diagnosis: No abnormality (complete cure)

In the examination data of the third examination, “complete cure” diagnosis data indicates that the doctor has diagnosed a complete cure for Patient A and that the follow-up has been completed. Even if diagnosis data indicating “complete cure” is not being registered, as long as the diagnosis data shows no abnormality and does not include “follow-up required”, the diagnosis data indicates that the doctor has diagnosed a complete cure for Patient A and that the follow-up has been completed.

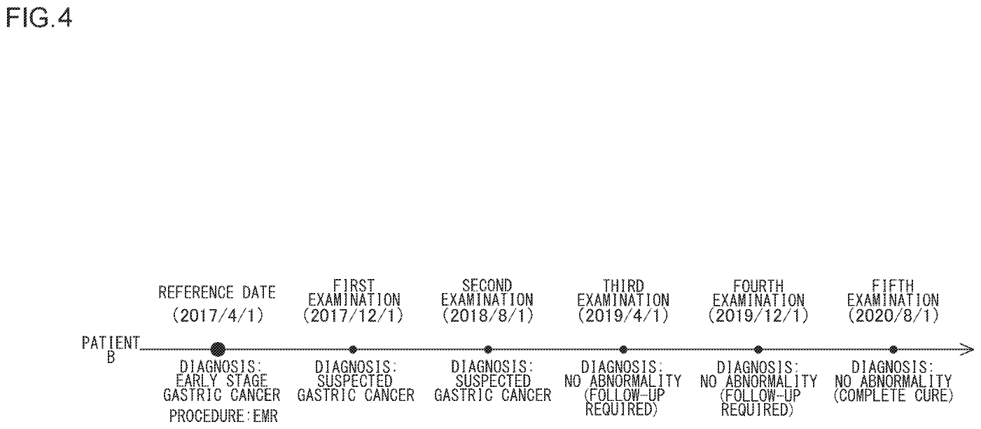

FIG. 4 shows a time series of examination data of Patient B. The first examination identification unit 22 identifies examination data that includes “early gastric cancer” diagnosis data and in which procedure data performed on the lesion is recorded. In the example shown in FIG. 4, the first examination identification unit 22 identifies examination data of Patient B whose examination date is 2017/4/1, and “EMR” is recorded in this examination data as the procedure performed on the lesion.

The second examination identification unit 24 uses the examination date (2017/4/1) of the examination data identified by the first examination identification unit 22 as a reference date and identifies respective pieces of examination data of multiple examinations performed on Patient B after the reference date. In the example shown in FIG. 4, the second examination identification unit 24 identifies examination data of the following five examinations.

(1) First Examination

Examination date: 2017/12/1

Diagnosis: Suspected gastric cancer

(2) Second Examination

Examination date: 2018/8/1

Diagnosis: Suspected gastric cancer

(3) Third Examination

Examination date: 2019/4/1

Diagnosis: No abnormality (follow-up required)

(4) Fourth Examination

Examination date: 2019/12/1

Diagnosis: No abnormality (follow-up required)

(5) Fifth Examination

Examination date: 2020/8/1

Diagnosis: No abnormality (complete cure)

As described above, the “complete cure” diagnosis data indicates that the doctor has diagnosed a complete cure for Patient B and that the follow-up has been completed. However, even if diagnosis data indicating “complete cure” is not being registered, as long as the diagnosis data shows no abnormality and does not include “follow-up required”, the diagnosis data indicates that the doctor has diagnosed a complete cure for Patient B and that the follow-up has been completed.

In the above, the time series of the examination data for Patient A shown in FIG. 3 and the time series of the examination data for Patient B shown in FIG. 4 indicate that the high-risk patients have completely cured after follow-up.

There also exists a time series of examination data indicating that the cancer has recurred, as a result of the first examination identification unit 22 identifying examination data that indicates that there is a possibility of cancer recurrence and the second examination identification unit 24 identifying examination data of an examination performed for the purpose of performing follow-up.

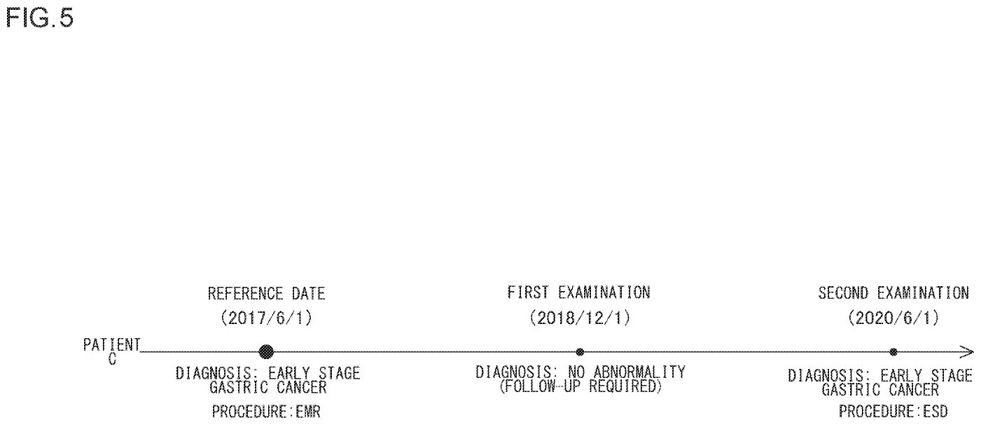

FIG. 5 shows a time series of examination data of Patient C. The first examination identification unit 22 identifies examination data that includes “early gastric cancer” diagnosis data and in which procedure data performed on the lesion is recorded. In the example shown in FIG. 5, the first examination identification unit 22 identifies examination data of Patient C whose examination date is Jun. 1, 2017, and “EMR” is recorded in this examination data as the procedure performed on the lesion.

The second examination identification unit 24 uses the examination date (2017/6/1) of the examination data identified by the first examination identification unit 22 as a reference date and identifies respective pieces of examination data of multiple examinations performed on Patient C after the reference date. In the example shown in FIG. 5, the second examination identification unit 24 identifies examination data of the following two examinations.

(1) First Examination

Examination date: 2018/12/1

Diagnosis: No abnormality (follow-up required)

(2) Second Examination

Examination date: 2020/6/1

Diagnosis: Early stage gastric cancer

Procedure: ESD

At the second examination, it is diagnosed that Patient C has recurrent cancer.

Patient C is a high-risk patient, and cancer recurrence can occur stochastically. In this examination, ESD is performed, and Patient C will therefore continue to be treated as a high-risk patient.

As described above, the search unit 20 identifies examination data that satisfies the conditions for identifying a patient as a high-risk patient and the subsequent examination data. The examination data of a large number of patients are accumulated in the recorder 4, and the search unit 20 identifies the examination data of a large number of high-risk patients and supplies materials for calculating the examination interval and the number of examinations to the analysis unit 30. The analysis unit 30 derives an appropriate examination interval and an appropriate number of examinations for a high-risk patient based on the examination data identified by the search unit 20.

The information acquisition unit 32 acquires, based on multiple pieces of examination data identified by the first examination identification unit 22 and the second examination identification unit 24, the period of time from the examination date (reference date) included in the examination data identified by the first examination identification unit 22 until the complete cure of the patient and the number of examinations performed until the complete cure of the patient.

Examination Data of Patient A

Referring to the time series of the examination data of Patient A, the information acquisition unit 32 acquires the period from the reference date until a diagnosis of a complete cure (follow-up period) and the number of examinations performed between the reference date and the diagnosis of a complete cure, as follows.

Follow-up period: 3 years

The number of examinations: 3

The follow-up period is acquired as the period of time from the reference date (2017/8/1) to the date of a complete cure diagnosis (2020/8/1). Further, the number of examinations is acquired as the number of examinations performed after the reference date and before the date of the complete cure diagnosis.

Examination Data of Patient B

Referring to the time series of the examination data of Patient B, the information acquisition unit 32 acquires the period of time from the reference date until a diagnosis of a complete cure (follow-up period) and the number of examinations performed between the reference date and the diagnosis of a complete cure, as follows.

Follow-up period: 3 years and 4 months

The number of examinations: 5

The follow-up period is acquired as the period of time from the reference date (2017/4/1) to the date of a complete cure diagnosis (2020/8/1).

Further, the number of examinations is acquired as the number of examinations performed after the reference date and before the date of the complete cure diagnosis.

As described above, the information acquisition unit 32 acquires the follow-up period and the number of examinations for each patient from the time series of examination data of cured high-risk patients.

The derivation unit 34 derives an appropriate examination interval I and an appropriate number N of examinations based on the follow-up period and the number of examinations acquired by the information acquisition unit 32 from the examination data of a plurality of the high-risk patients. The derivation unit 34 may derive the appropriate examination interval I and the appropriate number N of examinations by statistically processing the multiple follow-up periods and the multiple number of examinations acquired by the information acquisition unit 32, respectively.

For example, the derivation unit 34 may derive the median value of the multiple numbers of examinations acquired by the information acquisition unit 32 as an appropriate number N of examinations. As a method for the statistical process, not only the median value but also the mode value or the average value may be used, or another method may be even used. When using the average value, it is necessary to round off the decimal point and adjust the value so that the value becomes an integer.

The derivation unit 34 derives an appropriate follow-up period P in order to derive an appropriate examination interval I. The derivation unit 34 may derive the median value of the multiple follow-up periods acquired by the information acquisition unit 32 as an appropriate follow-up period P. As a method for the statistical process, not only the median value but also the mode value or the average value may be used, or another method may be even used. The derivation unit 34 derives the appropriate examination interval I (=P/N) by dividing (dividing) the derived follow-up period P by the derived number N of examinations.

The derivation unit 34 records the derived examination interval I and the number N of examinations in the statistical data recording unit 12 as statistical data of a high-risk patient who has been diagnosed with early-stage gastric cancer and who has undergone a procedure to remove the lesion. The above is based on the assumption that the high-risk patient has been diagnosed with early-stage gastric cancer. Alternatively, the search unit 20 may identify the examination data of a high-risk patient diagnosed with early-stage colorectal cancer, and the analysis unit 30 may derive the appropriate examination interval I and the appropriate number N of examinations for the high-risk patient diagnosed with early-stage colorectal cancer.

In the embodiment, a diagnosis of early cancer followed by a procedure performed to remove the lesion in an endoscopic examination is the condition for recognizing the patient as a high-risk patient, and the analysis unit 30 derives an appropriate examination interval I and an appropriate number N of examinations in a follow-up period. In another example, the analysis unit 30 may derive the appropriate examination interval I and the appropriate number N of examinations for each type of procedure.

In this case, the search unit 20 may separately perform a search for examination data of a high-risk patient who has been diagnosed with early-stage gastric cancer and has undergone EMR and a search for examination data of a high-risk patient who has been diagnosed with early-stage gastric cancer and has undergone ESD. In this way, the analysis unit 30 can separately derive an appropriate examination interval I and an appropriate number N of examinations for the high-risk patient who has undergone EMR and an appropriate examination interval I and an appropriate number N of examinations for the high-risk patient who has undergone ESD. The examination interval I and the number N of examinations are recorded in the statistical data recording unit 12 for each type of procedure.

The search unit 20 may identify examination data of a high-risk patient for each lesion condition such as the location and size of the lesion, and the analysis unit 30 may derive an appropriate examination interval I and an appropriate number N of examinations for the follow-up period for each lesion condition.

The derivation unit 34 may use examination data of patients with reoccurrence cancer to verify the appropriateness of the derived examination interval I. In the examination data of Patient C shown in FIG. 5, the examination interval is one year and six months with a diagnosis of cancer recurrence at the second examination. From this, it can be said that an examination interval shorter than one year and six months would have been preferred for early detection of cancer recurrence for Patient C. Therefore, if the derived examination interval I is longer than one year and six months, the derivation unit 34 may adjust the derived examination interval I to be shorter than one year and six months.

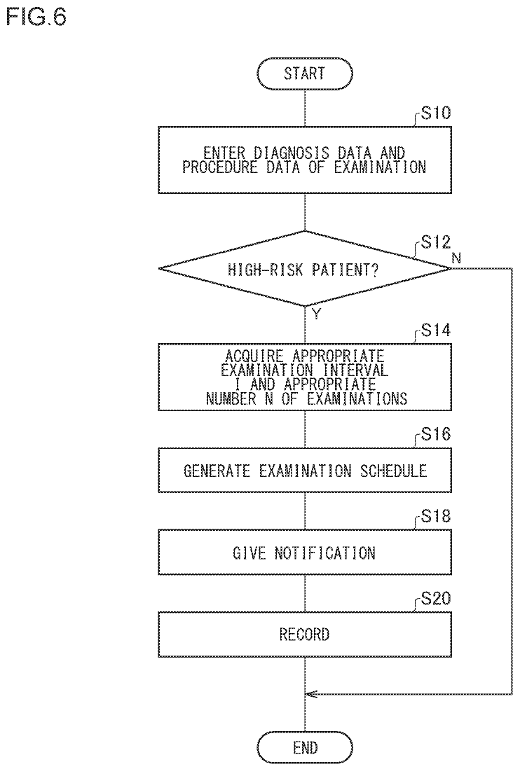

FIG. 6 is a flowchart for generating a high-risk patient's examination schedule. After the examination is completed, the display processing unit 50 displays an examination report entry screen on the display apparatus 3 based on an instruction from the doctor. The doctor operates the input unit 5 to enter diagnosis data, procedure data, etc., of an endoscopic examination into an examination report entry screen (S10). The examination schedule generation unit 40 determines whether or not the patient is a high-risk patient, i.e., whether or not the patient has a possibility of cancer recurrence, based on the entered diagnosis data and procedure data (S12). If the patient is not a high-risk patient (N in S12), the examination schedule generation unit 40 does not generate an examination schedule for the purpose of follow-up.

On the other hand, if the patient is a high-risk patient (Y in S12), the examination schedule generation unit 40 reads and acquires the examination interval I and the number N of examinations associated with the diagnosis data and the procedure data of the patient from the statistical data recording unit 12. If the medical system 1 does not have the statistical data recording unit 12, that is, if the examination interval I and the number N of examinations are not managed as statistical data, the examination schedule generation unit 40 may instruct the search unit 20 and the analysis unit 30 to process the derivation of the examination interval I and the number N of examinations so that the search unit 20 and the analysis unit 30 derive the examination interval I and the number N of examinations.

The examination schedule generation unit 40 generates an examination schedule for the follow-up of the patient using the examination interval I and the number N of examinations (S16). For example, if the examination interval I is one year and the number N of examinations is three, the examination schedule generation unit 40 generates an examination schedule with three examination dates per year starting from the examination date of an examination by which the patient is recognized as a high-risk patient. More specifically, if the examination date for the examination by which the patient is recognized as a high-risk patient is 2020/9/1, the examination schedule generation unit 40 schedules the first examination on 2021/9/1, the second examination on 2022/9/1, and the third examination on 2023/9/1.

The notification processing unit 46 notifies the doctor of information regarding the appropriate examination interval I and the appropriate number N of examinations (S18). The notification processing unit 46 may notify the doctor of a scheduled examination date generated by the examination schedule generation unit 40 or may directly notify the doctor of the examination interval I and the number N of examinations.

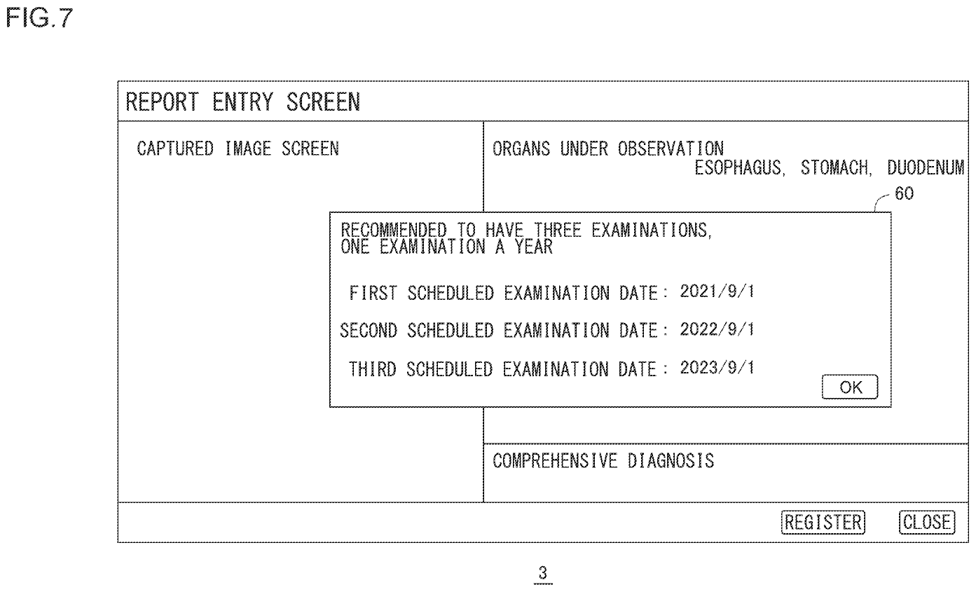

FIG. 7 is a diagram showing an example of a notification screen 60 displayed on a report entry screen. When diagnosis data and procedure data that meet the conditions for recognition of a high-risk patient are entered on the report entry screen, the notification processing unit 46 notifies the doctor of information regarding the examination interval I and the number N of examinations for the purpose of performing follow-up. In this example, the notification processing unit 46 displays a notification screen 60 including information regarding the examination interval I and the number N of examinations on the display apparatus 3. However, the doctor may be notified of this information by sound. The doctor records the patient's examination schedule information in the examination schedule information recording unit 14 in association with the patient ID (S20). The recording in the examination schedule information recording unit 14 may be performed automatically without the intervention of the doctor.

The above is an example of the notification processing unit 46 giving notification of information regarding the examination interval I and the number N of examinations during report entry by the doctor. Alternatively, the notification processing unit 46 may refer to the examination schedule information recorded in the examination schedule information recording unit 14 and provide notification regarding the examination schedule. More specifically, the notification processing unit 46 refers to the scheduled examination date recorded in the examination schedule information recording unit 14, and when the scheduled examination date is approaching, the notification processing unit 46 notifies a medical professional such as a doctor or nurse that the patient's scheduled examination date is approaching. As a result, the medical professional can inform the patient that the scheduled examination date is approaching and can suitably perform follow-up.

After the examination schedule is generated by the examination schedule generation unit 40, the patient's situation may change. For example, when a health checkup reveals hypertension, or when a family member develops cancer and the family history of cancer is updated. In these cases, the objective fact is that the risk of cancer recurrence is higher compared to when the examination schedule was generated. Therefore, when the patient information acquisition unit 42 acquires patient information regarding the risk of cancer recurrence, the examination schedule modification unit 44 may modify the examination schedule recorded in the examination schedule information recording unit 14 according to the acquired patient information. More specifically, the examination interval I is modified to be shortened while the number N of examinations is modified to be increased. For example, if the original examination interval I is one year and the number N of examinations is three, the examination schedule modification unit 44 may modify the examination interval I to nine months and the number N of examinations to four and update the scheduled examination date in the examination schedule information recording unit 14.

Described above is an explanation based on the embodiments of the present disclosure. These embodiments are intended to be illustrative only, and it will be obvious to those skilled in the art that various modifications to constituting elements and processes could be developed and that such modifications are also within the scope of the present disclosure.

In the embodiment, an explanation has been made for early gastric cancer than an appropriate examination interval I and an appropriate number N of examinations may be derived for each type of procedure. In the exemplary variation, the derivation unit 34 may derive a common examination interval I and a common number N of examinations for early stage gastric cancer regardless of the type of procedure, and the examination schedule generation unit 40 may adjust the common examination interval I and the common number N of examinations according to the details of the procedure performed on the patient. Alternatively, the examination schedule generation unit 40 may adjust the examination interval I and the number N of examinations according to the skill of the doctor who performed the procedure.

In the embodiment, the examination interval I and the number N of examinations are derived using as a starting point the examination date of the examination by which the patient is recognized as a high-risk patient. However, it is known that the possibility of cancer recurrence increases for patients whose cancer has recurred just like patient C shown in FIG. 5. Therefore, the search unit 20 may identify a time series of examination data for patients whose cancer has recurred, and the analysis unit 30 may derive an appropriate examination interval I and an appropriate number N of examinations for patients whose cancer has recurred.

What is claimed is:

1. An information processing apparatus that is connected to a recorder that holds a plurality of pieces of examination data including an examination date and diagnosis data of an endoscopic examination that has been performed, comprising:
one or more processors comprising hardware, wherein the one or more processors are configured to:
identify examination data including predetermined diagnosis data from among the plurality of pieces of examination data held in the recorder and extract the identified examination data as first data;
identify respective pieces of examination data of a plurality of examinations performed on a same patient after an examination date included in the first data from among the plurality of pieces of examination data held in the recorder and extract the identified examination data as second data;
acquire, as third data, a period from the examination date included in the first data until complete cure of the patient and the number of examinations performed until the complete cure of the patient based on the first data and the second data; and
derive an appropriate examination interval and an appropriate number of examinations based on the third data acquired from the plurality of pieces of examination data of the patient.

2. The information processing apparatus according to claim 1, wherein
the first data is examination data that indicates that there is a possibility of cancer recurrence.

3. The information processing apparatus according to claim 1, wherein the one or more processors are configured to:
give notification of information regarding the appropriate examination interval and the appropriate number of examinations.

4. The information processing apparatus according to claim 1, wherein the one or more processors are configured to:
generate an examination schedule for follow-up of the patient using the derived appropriate examination interval and the derived appropriate number of examinations.

5. The information processing apparatus according to claim 4, wherein the one or more processors are configured to:
acquire patient information regarding the risk of cancer recurrence;
modify the examination schedule according to the acquired patient information.

6. An information processing method comprising:
extracting, as first data, examination data including predetermined diagnosis data from a recorder holding a plurality of pieces of examination data including an examination date and diagnosis data of an endoscopic examination that has been performed;
extracting, as second data, respective pieces of examination data of a plurality of examinations performed on a same patient after an examination date included in the first data from the recorder;
acquiring, as third data, a period from the examination date included in the first data until complete cure of the patient and the number of examinations performed until the complete cure of the patient based on the first data and the second data; and
deriving an appropriate examination interval and an appropriate number of examinations based on the third data acquired from the plurality of pieces of examination data of the patient.

7. A non-transitory recording medium having a program that is executed by a computer recorded therein, the program comprising:
a processing instruction to extract, as first data, examination data including predetermined diagnosis data from a recorder holding a plurality of pieces of examination data including an examination date and diagnosis data of an endoscopic examination that has been performed;
a processing instruction to extract, as second data, respective pieces of examination data of a plurality of examinations performed on a same patient after an examination date included in the first data from the recorder;
a processing instruction to acquire, as third data, a period from the examination date included in the first data until complete cure of the patient and the number of examinations performed until the complete cure of the patient based on the first data and the second data; and
a processing instruction to derive an appropriate examination interval and an appropriate number of examinations based on the third data acquired from the plurality of pieces of examination data of the patient.
